(19) United States
(12) Patent Application Publication
(10) Pub. No.: US 2007/0039623 A1
(43) Pub. Date: Feb. 22, 2007

(54) Pathogenesis of the common headaches

(76) Inventor: Anuthep Benja-Athon, New York, NY (US)

Correspondence Address: ANUTHEP BENJA-ATHON, M.D.;Pain Management, Acupuncture Physical Therapy, 210 E 36th St., New York, NY, 10016, US

(21) Appl. No.: 11/205,859

(22) Filed: Aug. 18, 2005

(51) Int. Cl. A61B 19/00

(52) U.S. Cl. 128/898

(57) ABSTRACT

A method of describing and illustrating the pathogenesis of the common headaches. The method comprises processes of describing the initiating factors, events, sources or origins, and the roles of the peripheral and cranial nerves, spinal cord and brain and their peripheral and central networks in the pathogenesis of the headaches. The present invention also provides the best preventions and treatments of the headaches based on the pathogenesis.

### FIELD OF INVENTION

The pathogenesis of the common headaches.

### BACKGROUND OF THE INVENTION

Headaches are among the most common pain afflicting pain and suffering in human. It is estimated that 80% of men and women have headaches. Headaches are among the most common causes of disability in America.

Despite the above fact, the etiology or etiologies of the common headaches such as, but not limited to, tension-type headache, cluster headache, common migraine, classic migraine and related variants is or are unknown. Consequently, said headaches were not properly diagnosed and treated by most physicians and other healthcare providers and proper treatments are either unavailable or, if any, not properly applied.

Consequently, millions of patients and their families needlessly endure pain and suffering being caused by headaches. They, corporations and companies are wasting billions of dollars a year on improper treatments and ineffective drugs and devices. Said losses are further compounded by patients' high absenteeism and loss of wages and productivity.

### OJECTIVES OF THE INVENTION

The present invention provides a method of describing and illustrating the pathogenesis, pathophysiology and biomechanics of the common headaches.

The objectives of the present application are to describe and illustrate the origins and sources of said headaches, explain the pathogenesis, pathophysiology and biomechanics of said headaches, promote the understanding, prevention, optimal treatments of said headaches to immensely benefit patients and their families, physicians, companies and corporations and, therefore, to solve the above problems.

### SUMMARY OF THE INVENTION

The etiologies of the common headaches are unknown. Consequently, headaches remains poorly diagnosed and poorly or improperly treated by physicians and other healthcare providers.

The present invention provides a method of describing and illustrating the pathogenesis, neurophysiology and biomechanics of the common headaches. The method comprises a process of describing the initiating factors, events, sources or origins, and the roles of the peripheral nerves and cranial nerves, spinal cord, regions and areas of the brainstem and brain and their peripheral and central networks in the pathogenesis of said headaches and the related manifestations and syndromes. The present invention also provides the preventions and treatments of said headaches based on said pathogenesis and neurophysiology.

### DESCRIPTION OF THE PREFERRED EMBODIMENTS

It is understood that the common headaches are tension-type headache (also known as muscle-contraction headache, ordinary headache, stress headache, essential headache and idiopathic headache), cluster headache, migraine without aura or common migraine, migraine with aura or classic migraine henceforth collectively known as headaches.

The present invention is a method of describing and/or illustrating a pathogenesis or a neurophysiology of each said headache type of said headaches. The present invention provides a method of describing and/or illustrating the pathogenesis, neurophysiology and pathophysiology of said headaches and related manifestations and syndromes.

The present invention provides a process of describing and/or illustrating the initiating factor or event such as, but not limited to, an irritation, a distension, traction, displacement, an inflammation, a disruption of the blood supply to any of the structures infra, or any combination thereof in the pathogenesis of said headaches.

The present invention provides a process of describing and/or illustrating how said initiating factor or event causes change(s), derangement(s) or disorder(s) of any one component or a plurality of components of structures infra leading to or causing said headaches and relating manifestations and syndromes.

The present invention provides a process of describing and/or illustrating the source(s) or origin(s) of said headaches. The present invention provides a process of describing and/or illustrating said source(s) or origin(s) is/are, first, the cervical pain-producing and nonpain-producing structures such as posterior longitudinal ligament, anterior longitudinal ligament, cervical nerves such as, but not limited to, 1^{st}, 2^{nd} and 3^{rd} cervical nerves, joints including facet joints, muscles, bone, intervertebral discs, interspinous ligament, ligamentum flavum and dura mater.

Second, nerves such as, but not limited to 1^{st}, 2^{nd} and 3^{rd} cervical nerves, trigeminal nerve, glossopharyngeal nerve and vagus nerve are pain-producing structures. In addition, other structures are head and neck muscles, skin, subcutaneous tissue, skull periosteum, arteries, veins, eyes, ears, teeth, sinuses, membranes of the nasal cavity, oropharynx, venous sinuses, veins, arteries, dura, brain stem periaqueductal gray matter and sensory nuclei of the thalamus.

The present invention provides a process of describing and/or illustrating 1^{st}, 2^{nd} and 3^{rd} cervical spinal nerves, trigeminal nerve, glossopharyngeal nerve and vagus nerve as pain-producing structures and also as the nerves transmitting afferent signals including pain signals or nociceptive signals or impulses. The present invention further provides a process of describing the neural conduits such as, but not limited to, the cervical spinal nerves, trigeminal nerve, facial nerve and glossopharyngeal nerve, upper cervical spinal cord segments and brainstem for the autonomic nerves transmitting autonomic nerve signals including nociceptive signals or impulses.

The present invention provides a process of describing and/or illustrating the roles of nerves comprises a process of describing a role of a cervical nerve or a plurality of cervical nerves, i.e. 1st, 2^{nd} and/or 3^{rd} cervical nerve(s) and the sympathetic component of the autonomic nervous system in the neck in the production of said headaches. Furthermore, the present invention provides a process of describing a role of a nerve, such as, but not limited to, trigeminal nerve, glossopharyngeal nerve and vagus nerve and the autonomic nervous system in the brain in the production of said headaches. Said method also provides a process of describing the roles of certain brainstem areas or regions such as, but not limited, the reticular formation, hypothalamus and thalamus including their projections in the production of or involvement in said headaches.

The present invention provides a process of describing and/or illustrating the neural networks in the spinal cord and brain involving in said headaches—a process of describing and/or illustrating neuronal circuits in the peripheral nervous system and central nervous system relating to the cervical nerves, other spinal nerves, peripheral autonomic nervous system, spinal cord and its laminae such as laminae 1, 4, 5 and 6 in the dorsal horn, ascending spinal tracts, brainstem structures and areas such as, but not limited to the reticular formation and the thalamus and its nuclei, central autonomic nervous system—centers in the cerebrum, cerebellum, hypothalamus, midbrain, pons, medulla and pathways connecting all of these centers and pathways connecting these centers to the peripheral autonomic nervous system, Vth, VIIth, VIIIth, IXth and Xth cranial nerves and its nuclei, cerebrum, cerebellum, descending neural pathways and target areas and regions of said descending neural pathways in the central nervous system and target organs of the peripheral nerves.

For example, the present invention provides a process of describing and/or illustrating the modulated impulses passing inferiorly via conducting pathways that transmit impulses from the hypothalamus and midbrain, pons and medualla to the spinal cord where terminations of these conducting fibers synapse with the anterolateral and anterior horn cells whose axons form the peripheral sympathetic and somatic motor nerves, respectively. These become involve in segmental reflexes affecting organs such as, but not limited to, muscles, heart, vascular and visceral organs.

The present invention provides a means for selecting or using a treatment for any one of said headaches as the present invention elucidates the pathogenesis of said any and all members of said headaches and, therefore, provides the scientific basis on which appropriate medical care, treatments, medications and procedures can be designed, chosen and performed for the treatment of said headaches. For example, appropriate use of drugs and medications such as, but not limited to, local anesthetics and neurotoxins may be appropriately and optimally applied according to the present invention.

It is understood that the words “a pathogenesis” and “a neurophysiology” are singular and are applicable to each member of said headaches described herein. The plural form of said “a pathogenesis” and “neurophysiology” are “the pathogenesis” and “the neurophysiology” as used in the claims infra.

It is understood that the word “illustrating” used herein mean that illustrating or illustration using picture, figure, diagram, representation, a plurality of pictures, figures, diagrams, representations or the like or any combination thereof can and will be used to achieve of the goals and objectives of the present invention.

It is understood that the word “describing” used in the claims of the present invention infra also applies to illustrating or illustration using picture, figure, diagram, representation, a plurality of pictures, figures, diagrams, representations or the like or any combination thereof can and will be used to achieve of the goals and objectives of the present invention.

Although the preferred embodiments of this invention have been described, it will be appreciated by those skilled in the art and science that adaptations, variations, additions and detailed descriptions and/or illustrations may be made without departing from the spirit of the invention and the scope of the claims.

1. A method of describing the pathogenesis of the common headaches comprises:
a process of describing the initiating factors in the pathogenesis of said headaches;
a process of describing the events in the pathogenesis of said headaches;
a process of describing the sources or the origins of said headaches;
a process of describing the disorders of said sources or origins in the pathogenesis of said headaches;
a process of describing the roles of the nerves in the peripheral and central nervous systems in the pathogenesis of said headaches;
a process of describing the regions or areas of the spinal cord and brain involved in said headaches;
a process of describing the roles of the regions or areas of the spinal cord and brain in the pathogenesis of said headaches;
a process of describing the neural networks in the spinal cord and brain involved in said headaches; and
a process of describing the roles of the neural networks in the spinal cord and brain involved in the pathogenesis of said headaches.

2. The method of describing the pathogenesis according to claim 1 wherein said process of describing the sources or origins of said headaches comprises a process of describing the neck structures which are the sources or origins of said headaches.

3. The method of describing the pathogenesis according to claim 1 wherein said process of describing the sources or origins of said headaches comprises a process of describing the structures in the skull as the sources or origins of said headaches.

4. The process of describing the neck structures according to claim 2 comprises a process of describing the soft tissues of the neck as the sources or origins of said headaches.

5. The process of describing according to claim 4 includes a process of describing a cervical nerve as a source or an origin of said headaches.

6. The process of describing according to claim 4 includes the process of describing a plurality of cervical nerves as the sources or origins of said headaches.

7. The method according to claim 1 wherein said process of describing the roles of the nerves in the peripheral and central nervous systems contributing to said headaches comprises a process of describing a role of a cervical nerve.

8. The method according to claim 1 wherein said process of describing the roles of the nerves in the peripheral and central nervous systems contributing to said headaches comprises a process of describing a role of a cervical nerve and the sympathetic nerve component of the autonomic nervous system in the neck.

9. The method according to claim 1 wherein said process of describing the roles of the nerves in the peripheral and central nervous systems contributing to said headaches comprises a process of describing a role of a plurality of cervical nerves and the sympathetic component of the autonomic nervous system in the neck.

10. The method according to claim 1 wherein said process of describing the roles of the nerves in the peripheral and central nervous systems contributing to said headaches comprises a process of describing a role of a nerve and the autonomic nervous system in the brain.

11. The method according to claim 1 wherein said process of describing the roles of the nerves comprises a process of describing a role a cranial nerve.

12. The method according to claim 1 wherein said process of describing the roles of the nerves comprises a process of describing a role a plurality of cranial nerves.

13. The method according to claim 1 wherein said process of describing the roles of the nerves comprises a process of describing a role of the autonomic nervous system in the brainstem.

14. The method according to claim 1 wherein said process of describing the roles of the nerves comprises a process of describing a role of certain areas of the brainstem in the pathogenesis of said headaches.

15. The method according to claim 1 wherein said process of describing the roles of the nerves comprises a process of describing a role of certain areas of cerebrum in the pathogenesis of said headaches.

16. The method according to claim 1 wherein said process of describing the neural networks in the spinal cord and brain involving in said headache comprises a process of describing a plurality of neuronal circuits in the peripheral nervous system and central nervous system relating to spinal nerves including cervical nerves, cranial nerves, peripheral autonomic nervous system, spinal cord and its laminae, ascending spinal tracts, brainstem structures and areas, central autonomic nervous system, cerebrum, cerebellum, descending neural pathways and target areas and regions of said descending neural pathways in the central nervous system and target organs of the peripheral nerves.

17. A method of using the pathogenesis of the common headaches in the prevention and treatment of said headaches comprises:
a process of describing the initiating factors in the pathogenesis of said headaches;
a process of describing the events in the pathogenesis of said headaches;
a process of describing the sources or the origins of said headaches;
a process of describing the disorders of said sources or origins in the pathogenesis of said headaches;
a process of describing the roles of the nerves in the peripheral and central nervous systems in the pathogenesis of said headaches;
a process of describing the regions or areas of the spinal cord and brain involved in said headaches;
a process of describing the roles of the regions or areas of the spinal cord and brain in the pathogenesis of said headaches;
a process of describing the neural networks in the spinal cord and brain involved in said headaches; and
a process of describing the roles of the neural networks in the spinal cord and brain involved in the pathogenesis of said headaches.

18. A method of treating the common headaches based on the description of the pathogenesis of said headaches comprises:
a process of describing the initiating factors in the pathogenesis of said headaches;
a process of describing the events in the pathogenesis of said headaches;
a process of describing the sources or the origins of said headaches;
a process of describing the disorders of said sources or origins in the pathogenesis of said headaches;
a process of describing the roles of the nerves in the peripheral and central nervous systems in the pathogenesis of said headaches;
a process of describing the regions or areas of the spinal cord and brain involved in said headaches;
a process of describing the roles of the regions or areas of the spinal cord and brain in the pathogenesis of said headaches;
a process of describing the neural networks in the spinal cord and brain involved in said headaches; and
a process of describing the roles of the neural networks in the spinal cord and brain involved in the pathogenesis of said headaches.
